(19) United States
(12) Patent Application Publication
(10) Pub. No.: US 2025/0014765 A1
(43) Pub. Date: Jan. 9, 2025

(54) SYSTEMS AND METHODS FOR PREDICTING COVID 19 CASES AND DEATHS

(71) Applicant: Roche Molecular Systems, Inc., Pleasanton, CA (US)

(72) Inventors: Fernando Garcia-Alcalde, Basel (CH); Shimeng Huang, Toronto (CA); Elizabeth Little, San Jose, CA (US); Carsten Magnus, Zurich (CH); Anne-Marie Meyer, Bolligen (CH); Pascal Moritz, Lippstadt (DE); Christian Wohlfart, Penzberg (DE)

(73) Assignee: Roche Molecular Systems, Inc., Pleasanton, CA (US)

(21) Appl. No.: 18/891,815

(22) Filed: Sep. 20, 2024

Related U.S. Application Data
(63) Continuation of application No. PCT/EP2023/059139, filed on Apr. 6, 2023.
(60) Provisional application No. 63/362,818, filed on Apr. 11, 2022.

(51) Int. Cl. G16H 50/80; G06N 20/00; G16H 15/00

(52) U.S. Cl. CPC G16H 50/80; G16H 15/00; G06N 20/00

(57) ABSTRACT

A machine learning and/or deep learning framework forecasts epidemic or pandemic cases and deaths. Multiple open data sources relevant for the pandemic or epidemic evolution in a geographic area, such as the United States or another country or region, can be processed to extract a plurality of features, such as localized (i.e., county level, city level, regional level, province level, etc.) cases and deaths, demographics and socioeconomic factors, non-medical interventions, and mobility (i.e., from cell phone and/or GPS data). The learning can be used to predict future cases and deaths at localized levels and to recommend healthcare resources that may be needed.

### CROSS-REFERENCE TO RELATED APPLICATIONS

This application is a continuation of International Patent Application No. PCT/EP2023/059139 filed Apr. 6, 2023, which claims priority to U.S. Provisional Patent Application No. 63/362,818 filed Apr. 11, 2022, the disclosures of which are hereby incorporated by reference in their entirety.

### INCORPORATION BY REFERENCE

All publications and patent applications mentioned in this specification are herein incorporated by reference to the same extent as if each individual publication or patent application was specifically and individually indicated to be incorporated by reference.

### FIELD

Embodiments of the invention related generally to systems and methods of forecasting a disease in a population, and more particularly, to systems and methods of forecasting COVID-19 cases and deaths.

### BACKGROUND

Over the past year, the global Coronavirus Disease (COVID-19) has dominated and still influences our lives tremendously. Accurate and reliable forecasting of new cases and deaths at a local level continues to play an extremely important role to support decision-making to help first responders in healthcare, the public sector, and private organizations to be better prepared for what lies ahead.

### SUMMARY OF THE DISCLOSURE

Embodiments of the present invention relate generally to systems and methods of forecasting a disease in a population, and more particularly, to systems and methods of forecasting COVID-19 cases and deaths.

In one embodiment, a machine learning and/or deep learning framework was constructed and used to forecast COVID-19 cases and deaths. Multiple data sources from the respective provider relevant for the pandemic evolution in a geographic area, such as the United States or another country or region, can be processed to extract a plurality of features, such as localized (i.e., county level, city level, regional level, province level, etc.) COVID cases and deaths, demographics and socioeconomic factors, non-medical interventions, and mobility (i.e., from cell phone and/or GPS data).

In some embodiments, a generalizable and automated machine learning based modelling pipeline can be used to forecast daily cases and deaths at the US county-level. This framework is extensible and transferable to other countries (i.e., China, Japan, Great Britain, Spain, Italy, Germany, Switzerland, India, Canada, Australia, South Korea, Taiwan, Brazil, Mexico, South Africa, etc.) and regions (i.e., Europe, North America, South America, Asia, Middle East, Africa, etc.) worldwide. The outcomes of this framework was benchmarked and compared to current state-of-the-art US models, such as the CDC ensemble model and Google Cloud COVID-19 Public Forecasts.

In some embodiments, a method of forecasting COVID-19 related cases and/or deaths at a localized level is provided. The method includes obtaining data from a plurality of online databases; preprocessing the obtained data; extracting a plurality of feature vectors from the preprocessed data, wherein the plurality of feature vectors comprises mobility data, stringency measures, COVID-19 cases and/or deaths data, and demographic data; training a machine learning model using the extracted features and preprocessed data; validating the trained machine learning model; and predicting future COVID-19 cases and/or deaths at the localized level using the validated machine learning model.

In some embodiments, the localized level is a county level.

In some embodiments, the method further includes allocating health care resources at the localized level based on the predicted future COVID-19 cases and/or deaths.

In some embodiments, the method further includes allocating COVID-19 testing supplies at the localized level based on the predicted future COVID-19 cases and/or deaths.

In some embodiments, the plurality of feature vectors further includes time related data.

In some embodiments, the time related features include day of the year and holidays.

In some embodiments, the plurality of feature vectors further includes socio-economic data.

In some embodiments, the mobility data includes cell phone data and/or gps data.

In some embodiments, the stringency measures include data on curfews, lockdowns, business closures, school closures, and masking.

In some embodiments, the machine learning model is an ensemble model.

In some embodiments, the ensemble model is XGBoost.

In some embodiments, the ensemble model includes a plurality of gradient boosted decision tree algorithms.

In some embodiments, the plurality of feature vectors further comprises vaccination data.

In some embodiments, the vaccination data includes the types of vaccines, efficacy data for the types of vaccines, and vaccine administration data.

In some embodiments, the plurality of feature vectors further includes a first derivative in a number of cases over a time period of at least one day, a stringency index, an effective reproduction number, a contact index, a daily number of cases, a location, a day of year cycle, and a number of people that travelled less than one mile over a rolling period of seven days.

In some embodiments, the stringency index is the Oxford stringency index.

In some embodiments, the location is a US State.

In some embodiments, the day of year cycle is encoded with sine and cosine transformations.

In some embodiments, the plurality of feature vectors are used within the first year of an epidemic or pandemic.

In some embodiments, the plurality of feature vectors further comprises a change in a number of cases over a period of three days, an effective reproduction number, a daily number of cases, a daily number of cases as determined by a seven day rolling average, a rate of change in a number of cases over a seven day period, a first derivative in a number of cases over a time period of at least one day, a percent of people fully vaccinated, and a day of the week cycle.

In some embodiments, the plurality of feature vectors are used after the first year of an epidemic or pandemic.

In some embodiments, the plurality of feature vectors are used after a vaccine has been developed and administered to at least one person.

In some embodiments, the day of the week cycle is encoded with a cosine transformation.

In some embodiments, a system for forecasting COVID-19 related cases and/or deaths at a localized level is provided. The system can include a processor programmed to receive data from a plurality of online databases; preprocess the received data; extract a plurality of feature vectors from the preprocessed data, where the plurality of feature vectors includes mobility data, stringency measures, spatial data, COVID-19 cases and/or deaths data, and demographic data; train a machine learning model using the extracted features and preprocessed data; validate the trained machine learning model; and predict future COVID-19 cases and/or deaths at the localized level using the validated machine learning model.

In some embodiments, the processor is further programmed to perform any of the steps recited herein.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1 is a block diagram illustrating one embodiment of a computer system configured to implement one or more aspects of the present invention.

FIG. 2 is a flow chart of one embodiment of a method of creating a machine learning model for forecasting COVID-19 cases and deaths.

FIG. 3A illustrates an embodiment of a system for predicting COVID-19 cases and deaths.

FIG. 3B illustrates an embodiment of a method for downloading and preprocessing data from one or more sources of data.

FIGS. 4, 5A, and 5B are diagrams that illustrate various aspects of an embodiment of a gradient boosted decision tree algorithm.

FIG. 6 illustrates an embodiment of a method of training and testing the machine learning model using a train-test split.

FIG. 7 illustrates two validations approaches that can be used to validate the trained machine learning model.

FIGS. 8A-8C illustrate comparisons of the forecasts of COVID-19 cases from the model described herein with the Center for Disease Control's (CDC) ensemble model and the Google Cloud COVID model to actual case data.

FIGS. 9A-9C illustrate comparisons of the forecasts of COVID-19 deaths from the model described herein with the Center for Disease Control's (CDC) ensemble model and the Google Cloud COVID model to actual case data.

### DETAILED DESCRIPTION

### Computer Hardware for Implementing Methods

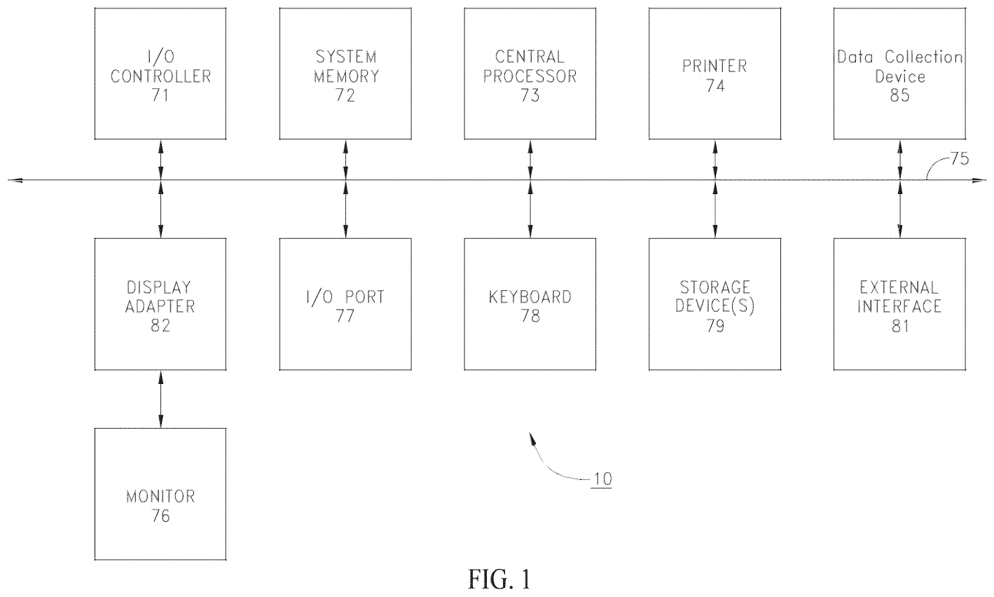

Embodiments of the machine learning and/or deep learning frameworks (i.e., pipelines) described herein can be implemented on a computer system. Any of the computer systems mentioned herein may utilize any suitable number of subsystems. Examples of such subsystems are shown in FIG. 1 in the computer system 10. In some embodiments, a computer system includes a single computer apparatus, where the subsystems can be the components of the computer apparatus. In other embodiments, a computer system can include multiple computer apparatuses, each being a subsystem, with internal components. A computer system can include desktop and laptop computers, tablets, mobile phones and other mobile devices. In some embodiments, a cloud infrastructure (e.g., Amazon Web Services), a graphical processing unit (GPU), etc., can be used to implement the disclosed techniques.

The subsystems shown in FIG. 1 are interconnected via a system bus 75. Additional subsystems such as a printer 74, keyboard 78, storage device(s) 79, monitor 76, which is coupled to display adapter 82, and others are shown. Peripherals and input/output (I/O) devices, which couple to I/O controller 71, can be connected to the computer system by any number of means known in the art such as input/output (I/O) port 77 (e.g., USB, FireWire®). For example, I/O port 77 or external interface 81 (e.g. Ethernet, Wi-Fi) can be used to connect the computer system 10 to a wide area network, such as the Internet, a mouse input device, or a scanner. The interconnection via system bus 75 allows the central processor 73 to communicate with each subsystem and to control the execution of a plurality of instructions from system memory 72 or the storage device(s) 79 (e.g., a fixed disk, such as a hard drive, or optical disk), as well as the exchange of information between subsystems. The system memory 72 and/or the storage device(s) 79 may embody a computer-readable medium. Another subsystem is a data collection device 85, such as a camera, microphone, accelerometer, temperature sensor, GPS device, and the like. Any of the data mentioned herein can be output from one component to another component and can be output to the user.

A computer system can include a plurality of the same components or subsystems, e.g., connected together by external interface 81 or by an internal interface. In some embodiments, computer systems, subsystems, or apparatuses can communicate over a network. In such instances, one computer can be considered a client and another computer a server, where each can be part of a same computer system. A client and a server can each include multiple systems, subsystems, or components.

Aspects of embodiments can be implemented in the form of control logic using hardware (e.g. an application specific integrated circuit or field programmable gate array) and/or using computer software with a generally programmable processor in a modular or integrated manner. As used herein, a processor includes a single-core processor, multi-core processor on a same integrated chip, or multiple processing units on a single circuit board or networked. Based on the disclosure and teachings provided herein, a person of ordinary skill in the art will know and appreciate other ways and/or methods to implement embodiments of the present invention using hardware and a combination of hardware and software.

Any of the software components or functions described in this application may be implemented as software code to be executed by a processor using any suitable computer language such as, for example, Java, C, C++, C #, Objective-C, Swift, or scripting language such as Perl or Python using, for example, conventional or object-oriented techniques. The software code may be stored as a series of instructions or commands on a computer-readable medium for storage and/or transmission. A suitable non-transitory computer-readable medium can include random access memory (RAM), a read-only memory (ROM), a magnetic medium such as a hard-drive or a floppy disk, or an optical medium such as a compact disk (CD) or DVD (digital versatile disk), flash memory, and the like. The computer-readable medium may be any combination of such storage or transmission devices.

Such programs may also be encoded and transmitted using carrier signals adapted for transmission via wired, optical, and/or wireless networks conforming to a variety of protocols, including the Internet. As such, a computer-readable medium may be created using a data signal encoded with such programs. Computer-readable media encoded with the program code may be packaged with a compatible device or provided separately from other devices (e.g., via Internet download). Any such computer-readable medium may reside on or within a single computer product (e.g. a hard drive, a CD, or an entire computer system), and may be present on or within different computer products within a system or network. A computer system may include a monitor, printer, or other suitable display for providing any of the results mentioned herein to a user.

### COVID-19 Prediction Method

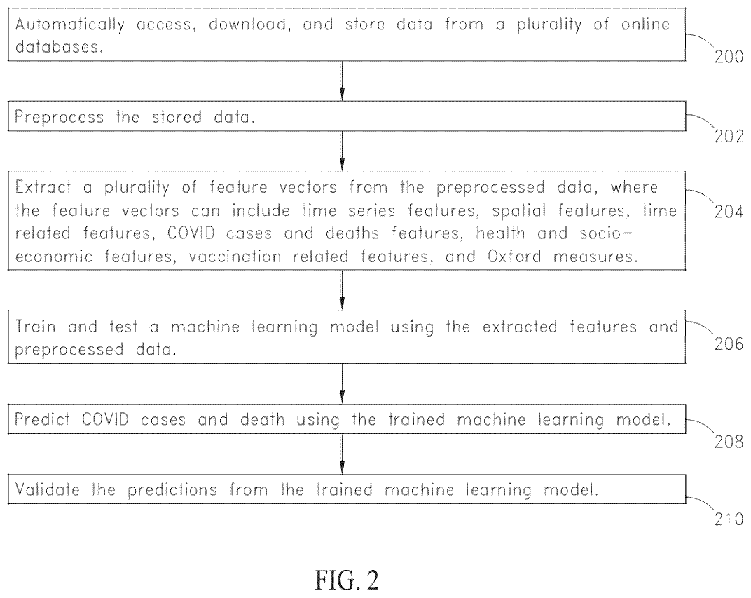

In some embodiments, as shown in FIG. 2, the proposed framework consists of five main parts: 1) automated collection 200 and preprocessing 202 of the identified data sources 2) feature engineering 204 to create time-series based predictors, 3) model selection, training, and testing 206 using predictors from previous 7-days, 14-days, 21-days or 28-days, as well as spatial-temporal cross validation approach including Bayesian hyperparameter tuning of the XGBoost model 4) model validation and final prediction 208, 210 of COVID cases and deaths, and 5) comparison to current COVID-19 forecasts and continuous monitoring of the model quality.

### 1. Automated Collection and Preprocessing of Data Sources

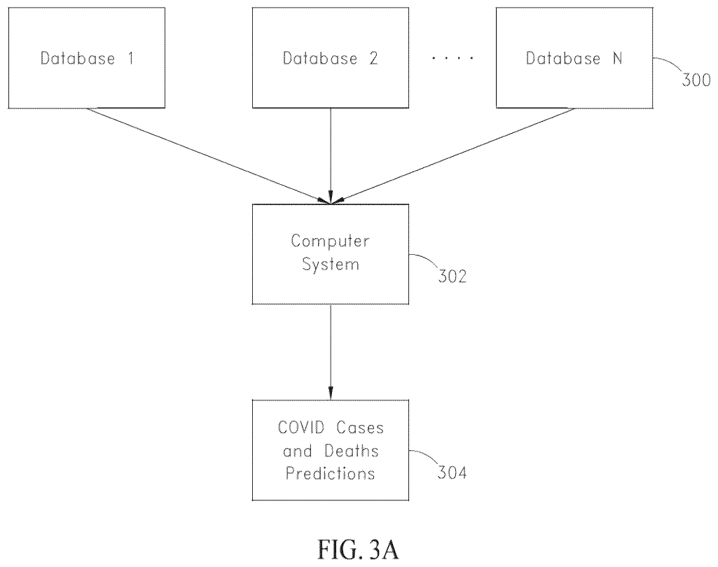

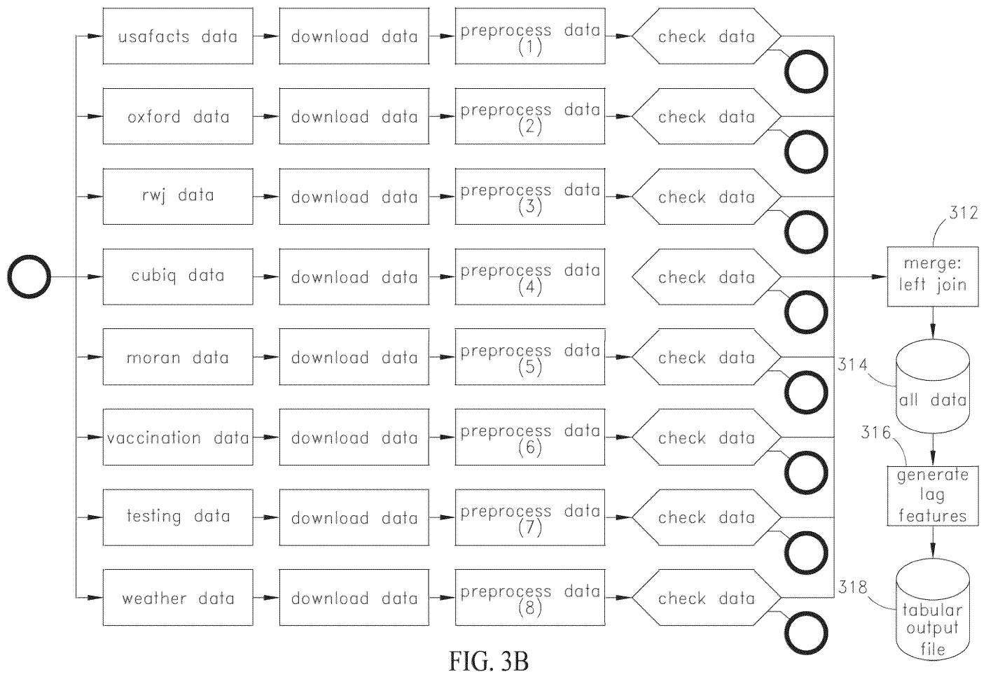

In some embodiments, as shown in FIG. 3, the data sources may include online databases 300 such as the COVID-19 Government Response Tracker released by the Univeristy of Oxford, RWJ County Health Rankings Data and Documentation released by Univeristy of Wisconsin Population Health Institute, County Health Rankings & Roadmaps 2020, US Covid-19 cases and deaths by state released by USAFACTS.org, Cuebig Mobility data released by CUBIQ, Effective Reproduction Number from Covidestim, Daily weather information from nearest station reported by National Oceanic and Atmospheric Administration, COVID-19 Vaccinations in the United States released by CDC, and/or COVID-19 Testing data released by Johns Hopkins University. Data from these databases can be automatically accessed, downloaded and collected or stored on a computer system 302 for further preprocessing.

Preprocessing of the data cleans and formats the data so that in can be input into the chosen machine learning model. Data preprocessing steps include transformation operations, county filtering, feature engineering, and target variable shift. In some embodiments, the filtering criteria for inclusion of a county into the dataset is optionally at least 9 cumulative cases and deaths during a minimum time period of at least 40 days for the model test and train period. In other embodiments, the filtering criteria can optionally require at least about 10, 20, 30, 40, 50, 60, 70, 80, 90, or 100 cumulative cases during a minimum time period of at least about 10, 20, 30, 40, 50, 60, 70, or 80 days for the model test and train period. In some embodiments, only the last 1, 2, 3, 4, 5, or 6 months are considered for the training model. Some steps may include cyclical feature generation and stair-wise encoding of ordinal features. Further, in some embodiments a data curation step can be implemented, where negative reported cases are corrected by subtracting the negative value from the previous time step.

For example, in some embodiments, the system and method can include data preprocessing steps as illustrated in FIG. 3B. The multiple sources of data 300 can be downloaded 306 from online databases as a csv, tsv or xls (x) file, for example, and can be preprocessed 308 by formatting the data into a tabular format or other data format for further processing by the system and method. Other preprocessing steps can include formatting dates, time data, area codes, zip codes, gps coordinates, and location data to a common standard. The data can also be checked 310 for consistency by, for example, dropping features above a predetermined or set missing rate (i.e., 50%, 60%, 70%, 80%, or 90%). The data can also be checked 310 for interval length by, for example, dropping datasets which are not complete or are outdated (i.e., older than a predetermined or set time such as 1, 2, 3, 4, 5, or 6 months, for example). The data can also be checked 310 for duplicates which can be dropped.

After the data is checked 310, the multiple datasets can be merged 312 into an aggregated dataset 314 by, for example, sequentially joining the data on “date” or other time feature and “area code” or other location feature. In some embodiments, the aggregated data 314 can be further processed to generate lag features 316.

To generate the lag features, for every 3 days (or other time interval such as 1, 2, 3, 4, 5, 6, or 7 days) in the range from 3 to 90 (or other time range such as 1 to 180 days), the method and system can: (1) generate one-period-shifted features for each feature, (2) decompose the one-period-shifted features via principal component analysis, FastICA (an algorithm for independent component analysis), or truncated singular value decomposition, and the like, and (3) use the number of components until at least 90% (or 95%, 85%, 80%, 75%, 70%, 65%, 60%, 55%, or 50%) of the variance is explained. Explained variance means that a certain explainabilty of the original features is achieved. E.g. 90% explained variance means that 90% of the original feature set can be explained with the new reduced feature set.

Additional lag features can be generated by (1) using the output components and performing a feature decomposition via, for example, principal component analysis, FastICA (an algorithm for independent component analysis), or truncated singular value decomposition, and the like, and (2) use the number of components until at least 90% (or 95%, 85%, 80%, 75%, 70%, 65%, 60%, 55%, or 50%) of the variance is explained. The lag features are temporal shift features to represent the time series characteristics.

After the lag features are generated, the data, which includes both the downloaded and preprocessed data and the generated lag features, can be stored in a tabular format as a tabular output file 318.

Other steps can include normalization, transformation, and feature extraction and selection, which are further described below.

### 2. Feature Engineering to Create Time-Series Based Predictors

In some embodiments, a plurality of feature vectors, which can be referred herein as simply features, can be extracted from the data obtained from the data sources using the computer system 302. In some embodiments, at least about 5, 10, 15, 20, 25, 30, 35, 40, 45, 50, 55, 60, 65, 70, 75, 80, 85, 90, 95 or 100 features can be extracted and used in the model. In other embodiments, no more than about 5, 10, 15, 20, 25, 30, 35, 40, 45, 50, 55, 60, 65, 70, 75, 80, 85, 90, 95, or 100 features can be used by the model.

In one embodiment, 9 time series features can be extracted from the daily COVID data. This includes temporal metrics including slope of cases/deaths or the first derivative based on smoothed time series curve (Savitzky-Golay filter). Further statistical metric are calculated (mean, standard deviation of 7d and 14d interval respectively) for cases and the 3d and 7d slope. Further, we added the effective reproduction number (Rt). Mobility features (9 features, i.e., gps data from mobile phones), 4 indices reflecting stringency measures (Stringency Index, Governmental Response Index, Containment Health Index, Economic Support Index) from Oxford, time related features (day of the year (DOY), holidays, week of year), Four Principal Components explaining 41 health and socio-economic features (n=41). In addition, weather (2 features), testing (2 features) and vaccination (4 features) were added from the aforementioned sources. In some embodiments, any combination of the above features can be used in the model.

For example, features that can be used by the method and system include the following, which can be county-wide features or other regional sized features: cumulative cases, daily cases (or other time interval such as 2, 3, 4, 5, 6, 7, 14, 21, 28 days, etc.), rate of change in the daily (or other time interval such as 2, 3, 4, 5, 6, 7, 14, 21, 28 days, etc.) cases over time, rolling mean or rolling standard deviation of cases over a period of time (i.e., 2, 3, 4, 5, 6, 7, 14, 21, or 28 days), total effective reproduction number (Rt), day of the year (sin and cos encoding for time variables can be used to account for the cyclic behavior of time), day of the week, weekday, weekend, holidays, measure of stringency measures (i.e., Oxford stringency index), measure of government response (i.e., Oxford government response index), measure of containment measures (i.e., Oxford containment index), measure of economic support (i.e., Oxford economic support index), premature deaths, poor or fair health, life expectancy, infant mortality, population, population density female, population below 18 years of age, population above 65 years of age, non-Hispanic Black, Hispanic, non-Hispanic white, other racial or ethnic group (i.e., Asian, Pacific Islander, Indigenous People, American Indian, and the like), living in rural area, living in urban area, not proficient in English (or other predominant local language), median household income, number or percentage that graduated from high school, number or percentage that attended at least some college, baseline unemployment rate, income inequality, social associations, violent crime, children eligible for free or reduced-price lunch, residential segregation-Black/white, residential segregation-non-white/white, homicides, diabetes prevalence, adult obesity, adult smoking, sexually transmitted infections, food insecurity, air pollution—particulate matter—PM 2.5, drinking water violations, households with severe housing problems, homeownership, severe housing cost, traffic volume, primary care physicians, preventable hospital stays, influenza vaccinations, coronavirus vaccinations, uninsured adults, uninsured children, mobility index (i.e., Cuebiq Mobility Index or other index that quantifies how far people move each day), mobility index (i.e., Cuebiq Mobility Index) average over a rolling time period (i.e., 2, 3, 4, 5, 6, 7, 14, 21, or 28 days), population that sheltered in place, average population that sheltered in place over a rolling time period (i.e., 2, 3, 4, 5, 6, 7, 14, 21, or 28 days), population that travelled less than 1 mile, population that travelled less than 10 miles, population that travelled less than 1, 2, 3, 4, 5, 6, 7, 8, 9, 10, 15, 20, 25, 30, 35, 40, or 50 miles over a rolling time period (i.e., 2, 3, 4, 5, 6, 7, 14, 21, or 28 days), population that travelled more than 1, 2, 3, 4, 5, 6, 7, 8, 9, 10, 15, 20, 25, 30, 35, 40, or 50 miles over a rolling time period (i.e., 2, 3, 4, 5, 6, 7, 14, 21, or 28 days), vaccination factors, proportion of the population fully vaccinated (i.e. number of people fully vaccinated per hundred), proportion of the population fully or partially vaccinated (i.e. total number of people with a least one dose of vaccine per hundred), increase in proportion of population fully vaccinated or partially vaccinated over a period of time (i.e., 1, 2, 3, 4, 5, 6, 7, 14, 21, or 28 days), vaccine efficacy for the above, testing factors, number or proportion of people testing positive, total number of combined tests increase, viral tests increase, weather factors, average temperature, rainfall, and differences of any of the above over a period of time (i.e., 1, 2, 3, 4, 5, 6, 7, 14, 21, or 28 days),

In some embodiments, any combination of the above features may be used. In some embodiments, fewer than about 100, 90, 80, 70, 60, 50, 40, 30, 20, or 10 features are used in the model.

In some embodiments, the model includes the following factors: the first derivative (i.e., the rate of change) in the number of cases over a time period (i.e., 1, 2, 3, 4, 5, 6, 7, 14, 21, or 28 days), the third principal component from the RWJ features, the Oxford stringency index, effective reproduction number at the current time (Rt) (can be calculated for each county, state, region, or other geographic subunit), the contact index that measures if two or more devices (e.g. mobile phones) come within a 50 feet range within a five minute time period over a rolling period of 7 days, second principal component from the RWJ features, daily number of cases, the US State (e.g. California, Texas, New York, etc.), day of year cycle encoding with sine and cosine transformation, and population that travelled less than 1 mile (or other distance as described herein) over a rolling period of 7 days. In some embodiments, these set of features are particularly suited for use in modeling the earlier stages of a pandemic or disease outbreak, such as during the first year of an outbreak.

In some embodiments that can be more suited for later stages of an outbreak, such as after the first year, the model can utilize a different feature set: three day slope in cases (i.e., the change in number of cases over a 3 day period), the effective reproduction number, the daily number of cases, the daily number of cases over a 7 day rolling average, rate of change in number of cases over a 7 day period), rate of change in the number of cases over a time period, percent of people fully vaccinated, and day of the week cycle encoding with cosine transformation.

### 3. Model Selection, Training, and Testing

In some embodiments, a machine learning algorithm and model can be implemented on the computer system 302 and used to forecast COVID-19 cases and deaths 304 on a localized basis.

Machine learning algorithms that can be used include, for example, ensemble based machine learning algorithms and/or decision tree based machine learning algorithms. Ensemble methods combine multiple learning algorithm algorithms into a single aggregate model. For example, a model based on a plurality of decision trees can be considered an ensemble method. Decision tree algorithms include (1) a basic decision tree based on a plurality of conditions; (2) a bootstrap aggregating or bagging algorithm is an ensemble algorithm that combines predictions from a plurality of decision trees through a majority voting approach; (3) a random forest algorithm is a bagging-based algorithm where only a subset of features are selected at random for each decision tree to build a plurality (i.e., a forest) of decision trees; (4) a boosting algorithm builds each decision tree sequentially by minimizing the errors from previous models (i.e., decision trees) while increasing (i.e. boosting) the weight or influence of high-performing models (i.e. decision trees); (5) a gradient boosting algorithm uses a gradient descent algorithm to minimize errors in sequential model (i.e. boosting algorithm in (4)); and (6) optimized gradient boosting algorithm (i.e. XGBoost) which may employ parallel processing, tree-pruning, handling missing values, and regularization to reduce overfitting and/or bias in gradient boosting algorithms. Any of these algorithms, alone or in combination, can be used. In other embodiments, a different machine learning algorithm can be used. In some embodiments, Lightgbm by Microsoft and Tabnet by Google can be used. In some embodiments, an ensemble model incorporating any combination of these models can be used. In some embodiments, the different models can be averaged, a mean taken, and/or weighted can be used. Elastic nets, Automatic Relevance Determination Regression and Epsilon-Support Vector Regression can also be used.

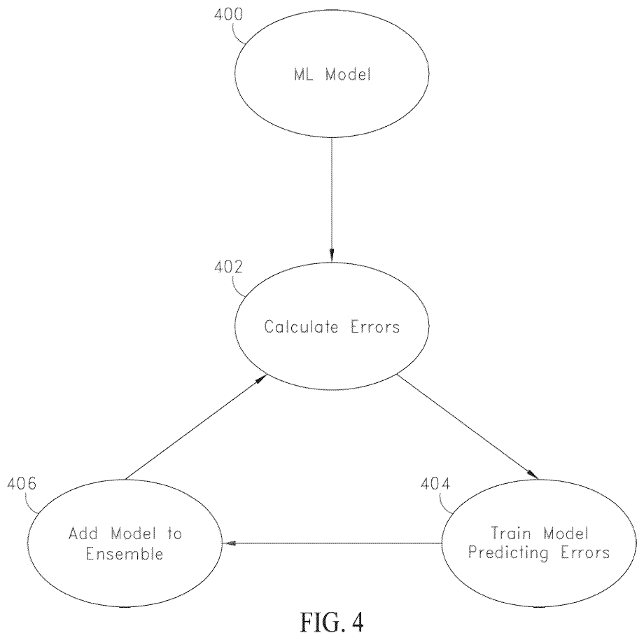

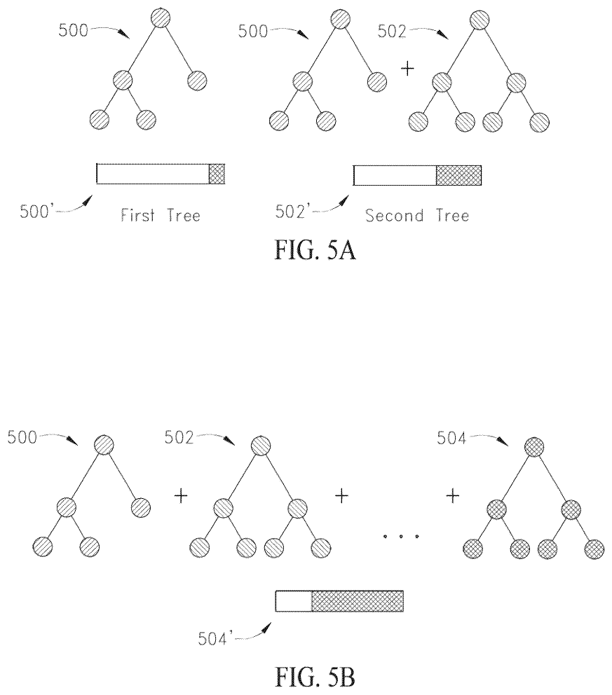

For example, in some embodiments a gradient boosting algorithm such as XGBoost can be used. XGBoost is an optimized variation of a gradient boosted decision tree algorithm, and is shown at a high level in FIG. 4. One of the primary differences between the random forest algorithm and a boosted decision tree algorithm is in how the models are trained. In the random forest model, all the trees are trained together as a group, while in a boosted decision tree model, the training and construction of the model occurs iteratively, as shown in FIG. 4. For example, in a boosted decision tree algorithm, at step 400 a first decision tree can be constructed and trained. Then in step 402, the error of the first decision tree can be determined. Depending on the type of error function or algorithm used to determine and minimize the error, the model can be considered a boosted decision tree, a gradient boosted decision tree, or an optimized gradient boosted decision tree such as XGBoost. For example, a gradient boosted decision tree algorithm uses a gradient descent algorithm to minimize the error. In step 404, a second decision tree can be constructed using the feedback from the error determination step and then trained. In step 406, the new decision tree can be added to the first decision tree to form an ensemble model. Steps 402, 404, and 406 can be repeated until the ensemble model contains a selectable or predetermined number of trees, or the ensemble model satisfies one or more performance criteria, such as not exceeding a selectable or predetermined level of error. FIGS. 5A and 5B illustrate the iterative addition of decisions trees as described above in connection with FIG. 4, starting with a first tree 500, then adding a second tree 502, and then adding a N-th tree 504. Additionally, the error of the new decision tree is illustrated in the error bar 500′, 502′, 504′ below the respective decision tree 500, 502, 504, with the ratio of the lighter portion to the darker portion of the bar representing the magnitude of the error.

In some embodiments, the XGBoost model can be used to analyze county-wide data to predict Covid-19 cases and deaths on a daily basis, weekly basis, biweekly basis, or monthly basis. Hyperparameter tuning can also be performed on a regular basis.

Table 1 illustrates examples of some tuned hyperparameters that can be used by the models.

| Method | Parameter | Type | Domain |
|---|---|---|---|
| xgboost | learning_rate | continuous | 0.001, 0.8 |
| xgboost | gamma | continuous | 0, 30 |
| xgboost | max_depth | continuous | 5, 100 |
| xgboost | n_estimators | continuous | 30, 400 |
| xgboost | min_child_weight | continuous | 1, 20 |
| Elastic net | alpha | continuous | 0.01, 10 |
| ARD | alpha_1 | continuous | 0.00001, 0.3 |
| ARD | alpha_2 | continuous | 0.00001, 0.3 |
| ARD | lambda_1 | continuous | 0.00001, 0.3 |
| ARD | lambda_2 | continuous | 0.00001, 0.3 |
| SVR | degree | continuous | 3, 7 |
| SVR | tol | continuous | 0.0001, 0.2 |
| SVR | C | continuous | 0.01, 10 |
| lightgbm | num_leaves | continuous | 30, 90 |
| lightgbm | reg_alpha | continuous | 0.00001, 10 |
| lightgbm | reg_lambda | continuous | 0.00001, 10 |
| lightgbm | max_depth | continuous | 0, 30 |
| lightgbm | n_estimators | continuous | 30, 400 |
| lightgbm | min_child_weight | continuous | 1, 20 |
| Tabnet | patience | continuous | 100, 1000 |
| Tabnet | max_epochs | continuous | 500, 10000 |

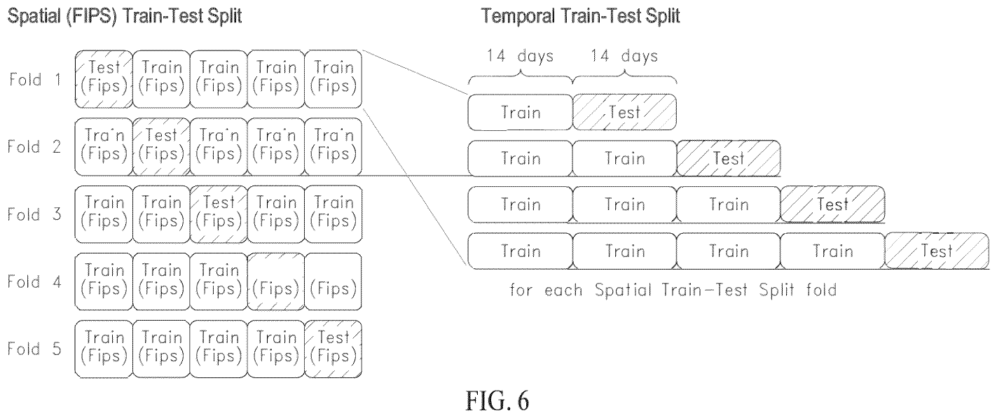

In some embodiments, the XGBoost model can be trained using a spatial-temporal cross-validation approach as shown in FIG. 6. For each spatial train-test split fold, a second layer temporal train-test split was generated. For example, in some embodiments, within the first layer all areas are split into one train dataset and one test dataset randomly. Within this first layer every dataset has distinct areas. In the second layer, every dataset is split into training and testing again by a time-series split on a 14-day or 28-day interval (or other time interval (i.e., 7, 14, 21, 28, 35, 42, 49, 56, 63, or 70 days). This second layer train test split is done as long as no more described interval can be done. Next this approach is iterated at least 20 times (or at least 5, 10, 15, 20, 25, 30, 35, 40, 45, or 50 times), and differs only by the first layer and the random assignments of all areas to training and testing. The term “FIPS” in FIG. 6 stands for the Federal Information Processing Standard, which is a five digit code that uniquely represents counties in the United States.

### 4. Model Validation and Predictions of COVID Cases and Deaths

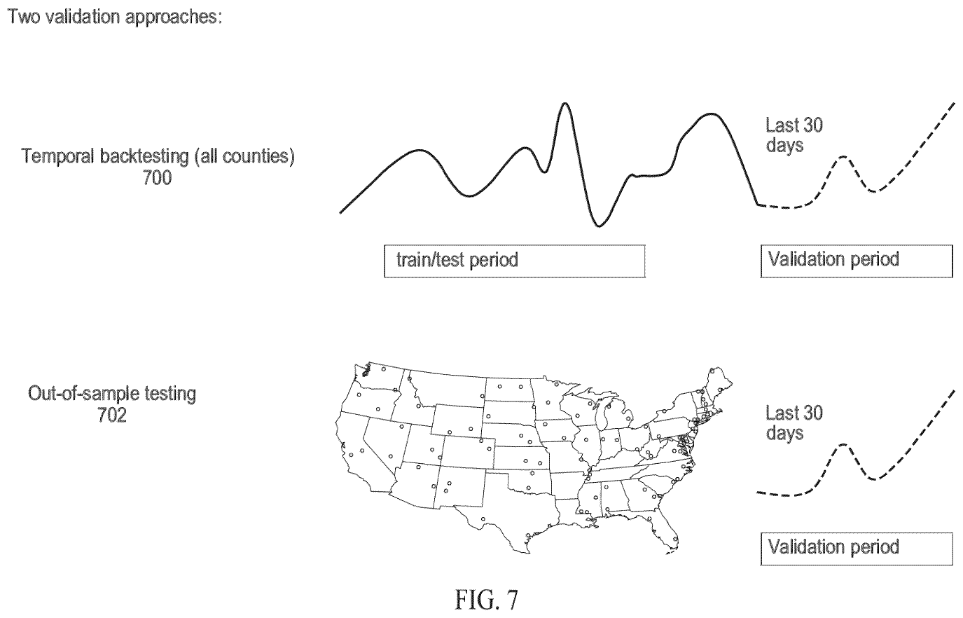

In some embodiments, the machine learning model can be validated using one or more approaches, as shown in FIG. 7. One validation method 700 can be performed by temporal backtesting of all counties. The dataset from the databases can be divided into two portions by time periods, with one time period used as the train and test period and the second time period as a validation period. In some embodiments, the validation period can be the last 30 days, while other embodiments can be less than 30 days, such as the last 7, 14, 21, or 28 days. In other embodiments, the validation period can be greater than 30 days, such as the last 45, 60, 75, 90, 105, 120, 135, 150, 165, or 180 days.

A second validation method 702 can be performed using out-of-sample testing. This method can also have a validation period like that described above, such as the last 30 days.

In some embodiments, training and testing are done to generate the model and tune the hyperparameters. Validation can be done using the last 30 days of every time series and can be used to validate the goodness of fit outside the timeseries that is used for testing and training. This is an example of using temporal backtesting. A second approach that can be used is called spatial backtesting which takes the whole timeseries for training and testing and leaves out two counties by state. In some embodiments, a combination that uses both temporal backtesting and spatial backtesting can be used. This can be evaluated by using statistical metrics which calculate the difference between actual and predicted values, e.g. rmse, nrmse, mape, mac.

In some embodiments, the model only predicts the number of cases and does not predict the number of deaths. In some embodiments, the model predicts both the number of cases and the number of deaths. In other embodiments, the model predicts only the number of deaths.

### 5. Comparison to Current COVID-19 Forecasts and Continuous Monitoring of the Model Quality

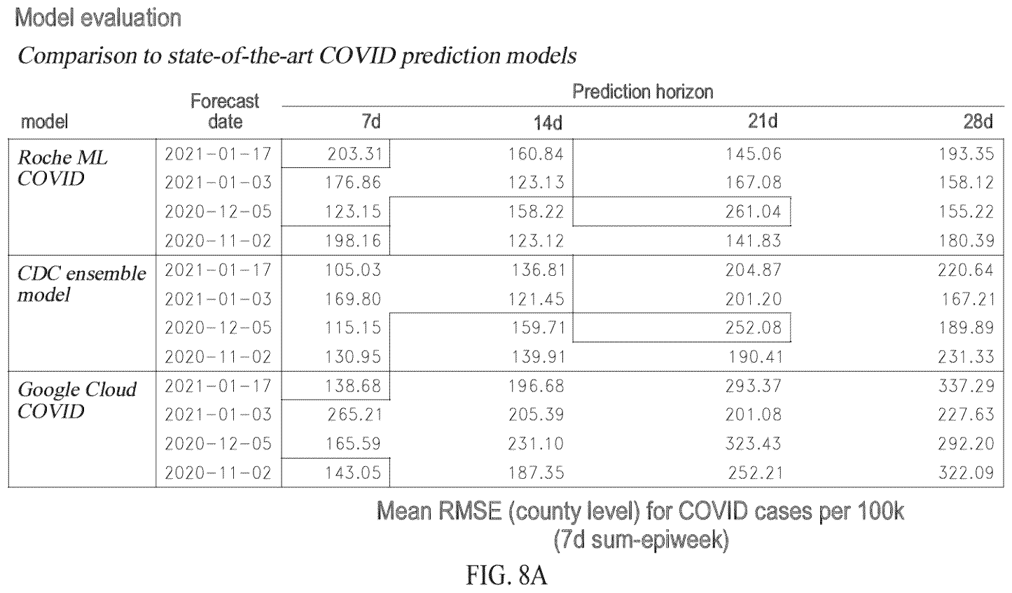

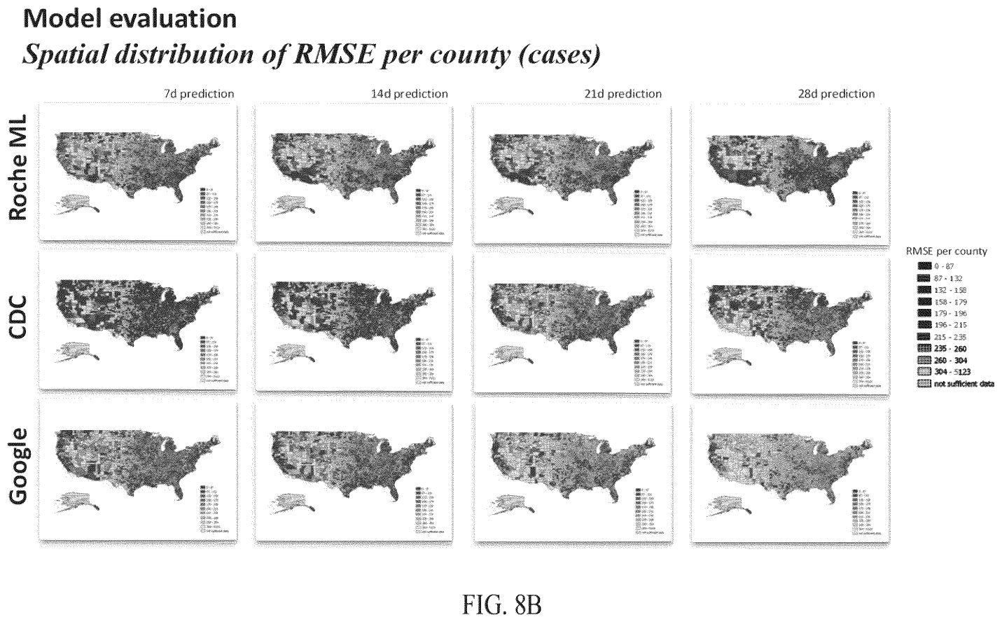

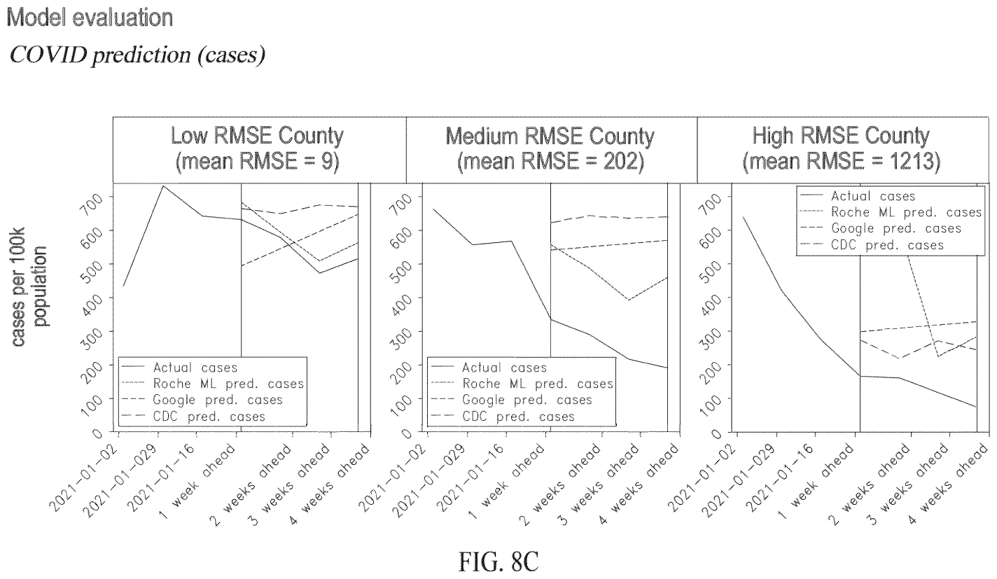

FIGS. 8A-8C illustrate comparisons of the forecasts of COVID-19 cases from the model described herein with the Center for Disease Control's (CDC) ensemble model and the Google Cloud COVID model to actual case data.

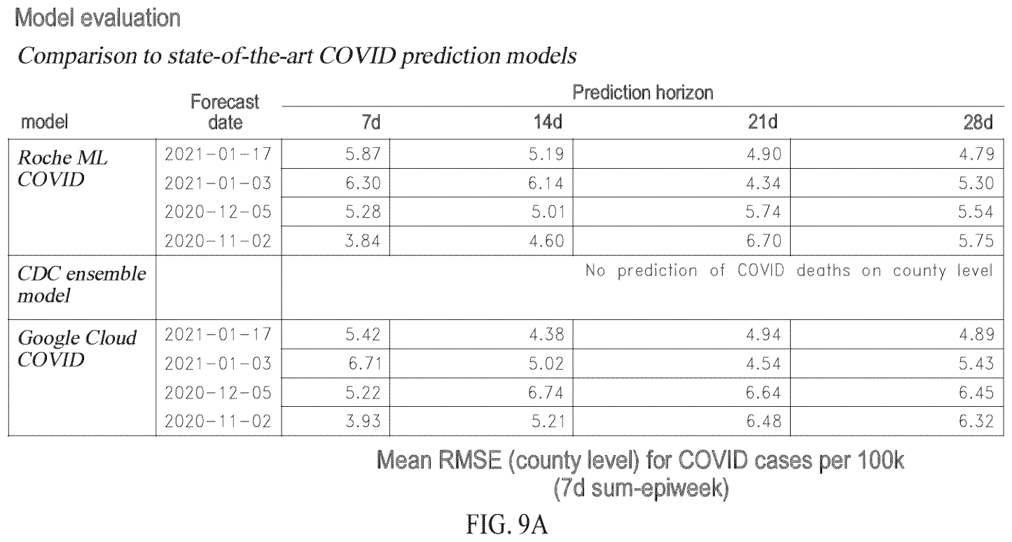

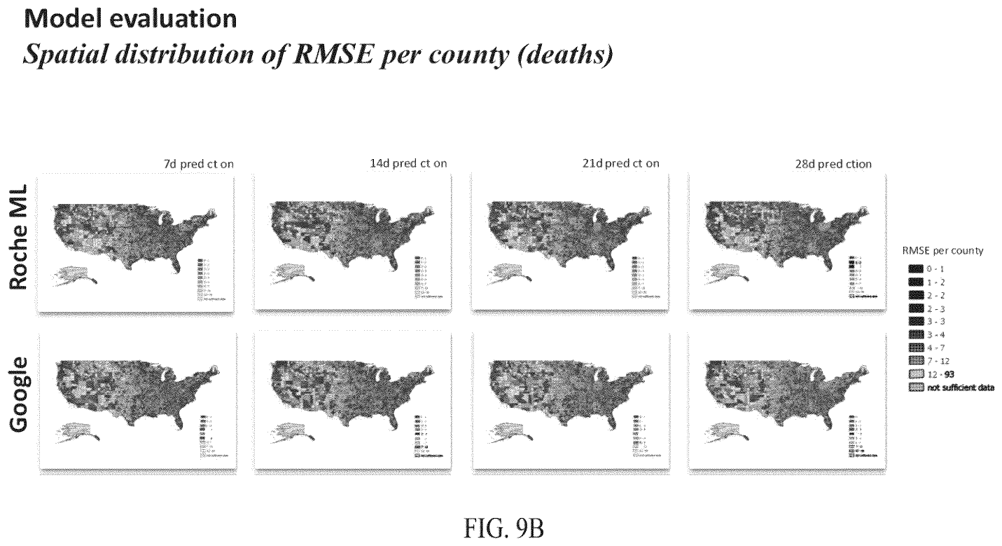

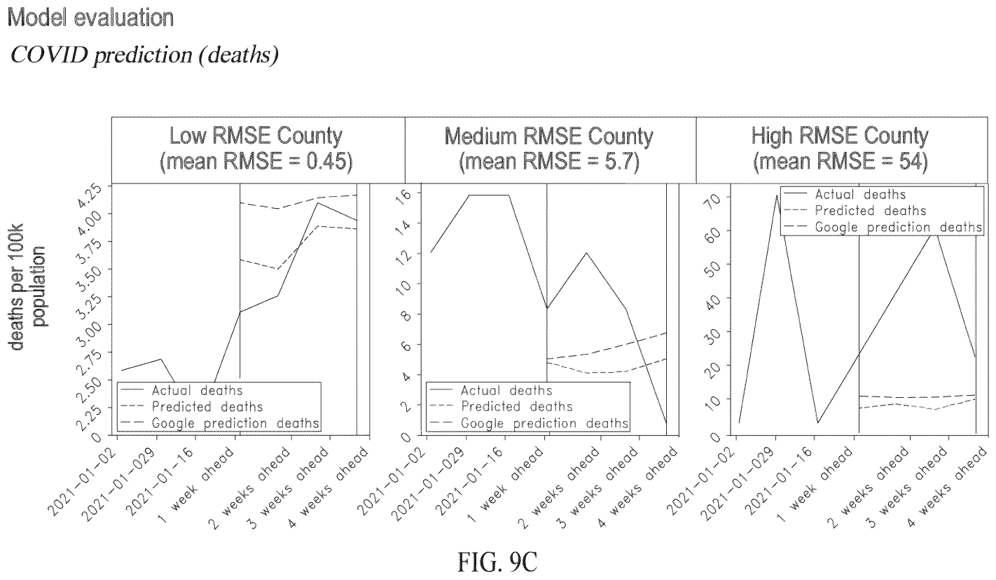

FIGS. 9A-9C illustrate comparisons of the forecasts of COVID-19 deaths from the model described herein with the Center for Disease Control's (CDC) ensemble model and the Google Cloud COVID model to actual case data.

Comparison with the CDC and Google models shows that the model described herein generally outperforms the other models on the long term forecast (3-4 week timeframe).

Improved ability to forecast COVID-19 cases and deaths allows health care resources to be better allocated by providing a lead time of 1, 2, 3, or 4 or more weeks advance notice of a spike in cases and deaths. For example, hospitals can obtain extra beds, ventilators, masks, gloves, gowns, medicines, oxygen, to meet the anticipated demand. Similarly, SARS-COV-2 testing supplies can also be allocated and delivered to future hotspots in advance of the increase in cases. In addition, public health care measures, such as curfews, lockdowns, business closures, school closures, and/or masking, can be implemented to reduce or prevent the future spike in cases.

Any of the methods described herein may be totally or partially performed with a computer system including one or more processors, which can be configured to perform the steps. Thus, embodiments can be directed to computer systems configured to perform the steps of any of the methods described herein, potentially with different components performing a respective step or a respective group of steps. Although presented as numbered steps, steps of methods herein can be performed at the same time or in a different order. Additionally, portions of these steps may be used with portions of other steps from other methods. Also, all or portions of a step may be optional. Additionally, any of the steps of any of the methods can be performed with modules, units, circuits, or other means for performing these steps.

The specific details of particular embodiments may be combined in any suitable manner without departing from the spirit and scope of embodiments of the invention. However, other embodiments of the invention may be directed to specific embodiments relating to each individual aspect or specific combinations of these individual aspects.

The above description of example embodiments of the invention has been presented for the purposes of illustration and description. It is not intended to be exhaustive or to limit the invention to the precise form described, and many modifications and variations are possible in light of the teaching above.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A computer-implemented method of forecasting epidemic or pandemic related cases and/or deaths at a localized level, the method comprising:
obtaining data from a plurality of online databases;
preprocessing the obtained data;
extracting a plurality of feature vectors from the preprocessed data, wherein the plurality of feature vectors comprises mobility data over a rolling time period, vaccination data, stringency measures, epidemic or pandemic cases and/or deaths data, and demographic data;
training a machine learning model using the extracted features and preprocessed data;
validating the trained machine learning model; and
predicting future epidemic or pandemic cases and/or deaths at the localized level using the validated machine learning model.

2. The method of claim 1, further comprising recommending levels of health care resources at the localized level based on the predicted future epidemic or pandemic cases and/or deaths.

3. The method of claim 1, further comprising recommending levels of epidemic or pandemic testing supplies at the localized level based on the predicted future epidemic or pandemic cases and/or deaths.

4. The method of claim 1, wherein the plurality of feature vectors further comprises calendar related data.

5. The method of claim 1, wherein the plurality of feature vectors further comprises socio-economic data.

6. The method of claim 1, wherein the feature vectors comprise weather data.

7. The method of claim 1, wherein the mobility data comprises cellular network data and/or gps data.

8. The method of claim 1, wherein the stringency measures comprise data on curfews, lockdowns, business closures, school closures, and masking.

9. The method of claim 1, wherein the machine learning model comprises an optimized gradient boosting algorithm.

10. The method of claim 9, wherein the machine learning model comprises a plurality of gradient boosted decision tree algorithms.

11. The method of claim 1, wherein the vaccination data comprises types of vaccines, efficacy data for the types of vaccines, and vaccine administration data.

12. The method of claim 1, wherein the plurality of feature vectors further comprises a first derivative in a number of cases over a time period of at least one day, a stringency index, an effective reproduction number, a contact index, a daily number of cases, a location, a calendar subcycle, and a number of people that travelled less than one mile over a rolling period of seven days.

13. The method of claim 12, wherein the calendar subcycle is encoded with at least one of sine and cosine transformations.

14. The method of claim 1, wherein the plurality of feature vectors further comprises a change in a number of cases over a period of three days, an effective reproduction number, a daily number of cases, a daily number of cases as determined by a seven day rolling average, a rate of change in a number of cases over a seven day period, a first derivative in a number of cases over a time period of at least one day, a percent of people fully vaccinated, and a day of the week cycle.

15. A system for forecasting epidemic or pandemic related cases and/or deaths at a localized level, the system comprising:
one or more processors programmed to:
receive data from a plurality of online databases;
preprocess the received data;
extract a plurality of feature vectors from the preprocessed data, wherein the plurality of feature vectors comprises mobility data over a rolling time period, stringency measures, spatial data, vaccination data, epidemic or pandemic cases and/or deaths data, and demographic data;
train a machine learning model using the extracted features and preprocessed data;
validate the trained machine learning model; and
predict future epidemic or pandemic cases and/or deaths at the localized level using the validated machine learning model.

16. The system of claim 15 wherein the one or more processors are programmed to:
recommend levels of health care resources at the localized level based on the predicted future epidemic or pandemic cases and/or deaths.

17. The system of claim 15 wherein the one or more processors are programmed to:
recommend levels of epidemic or pandemic testing supplies at the localized level based on the predicted future epidemic or pandemic cases and/or deaths.

18. The system of claim 15 wherein the plurality of feature vectors further comprises a first derivative in a number of cases over a time period of at least one day, a stringency index, an effective reproduction number, a contact index, a daily number of cases, a location, a calendar subcycle, and a number of people that travelled less than one mile over a rolling period of seven days.

19. The system of claim 15 wherein the feature vectors comprise weather data.

20. A computer readable medium storing instructions for causing one or more processors to:
receive data from a plurality of online databases;
preprocess the received data;
extract a plurality of feature vectors from the preprocessed data, wherein the plurality of feature vectors comprises mobility data over a rolling time period, stringency measures, spatial data, epidemic or pandemic cases and/or deaths data, vaccination data, and demographic data;
train a machine learning model using the extracted features and preprocessed data;
validate the trained machine learning model; and
predict future epidemic or pandemic cases and/or deaths at the localized level using the validated machine learning model.
